(19) United States
(12) Patent Application Publication
(10) Pub. No.: US 2026/0335171 A1
(43) Pub. Date: Oct. 29, 2026

(54) ADAPTABLE COMPRESSION SYSTEM FOR TREATING HYPOTENSION CRISIS EVENTS AND RELATED DEVICES AND METHODS

(71) Applicant: Alexandria Biotech, LLC, Minneapolis, MN (US)

(72) Inventor: Ahmed Selim, Colleyville, TX (US)

(21) Appl. No.: 19/733,764

(22) Filed: Jul. 7, 2026

Related U.S. Application Data
(63) Continuation-in-part of application No. 19/213,498, filed on May 20, 2025.
(60) Provisional application No. 63/649,748, filed on May 20, 2024.

(51) Int. Cl. A61H 7/00; A61F 13/08

(52) U.S. Cl. CPC A61H 7/001; A61F 13/08; A61H 2201/0103; A61H 2201/1238; A61H 2201/165; A61H 2201/5058; A61H 2205/106; A61H 2205/108; A61H 2230/255; A61H 2230/505

(57) ABSTRACT

The disclosed system, and related devices and methods, relate to the automatic compression of the body parts of a patient to treat and prevent symptoms of orthostatic hypotension. The system, devices, and methods can monitor the position, temperature, and/or blood flow of the patient to only employ compression when an orthostatic hypotension event occurs.

### CROSS-REFERENCE TO RELATED APPLICATION(S)

This application is a continuation-in-part application of U.S. patent application Ser. No. 19/213,498, entitled “ADAPTABLE COMPRESSION SYSTEM FOR TREATING HYPOTENSION CRISIS EVENTS AND RELATED DEVICES AND METHODS,” filed May 20, 2025, which claims priority under 35 U.S.C. § 119(e) to U.S. Provisional Application No. 63/649,748, entitled “ADAPTABLE COMPRESSION SYSTEM FOR TREATING HYPOTENSION CRISIS EVENTS AND RELATED DEVICES AND METHODS,” filed May 20, 2024, both of which are hereby incorporated by reference in their entirety for all purposes.

### TECHNICAL FIELD

The disclosure relates to medical treatment, generally, and systems to treat orthostatic hypotension, specifically.

### BACKGROUND

Various medical conditions are associated with an inability to or difficulty in returning blood from extremities to the heart. Such medical conditions include or are related to orthostatic hypotension, postural orthostatic tachycardia syndrome, chronic venous stasis, autonomic dysfunction, peripheral nerve damage, and conditions associated with intravascular hypovolemia. Current treatments have limitations in effectiveness, immediateness, and tolerability. There is a need for improved systems, methods, and devices to manage orthostatic hypotension and enhance patient outcomes.

### BRIEF SUMMARY

Described herein are various implementations relating to a system to provide variable and adaptive compression for the treatment of a hypotension crisis event. The system, through various implementations, may be applied to different areas of the body of a patient, as may be needed. The system may incorporate automatic compression technologies with various working principles to provide optimal treatment for any particular patient.

In Example 1 a system for treating a hypotension crisis event comprises a sleeve shaped to be worn over a body part of a patient, a tension device disposed on the sleeve, and one or more sensors in operable communication with the tension device.

Example 2 relates to the system of any of Examples 1 and 3-15, wherein the tension device comprises an inflatable cuff and a pump unit.

Example 3 relates to the system of any of Examples 1-2 and 4-15, wherein the pump unit activates upon receiving a communication from the one or more sensors which causes the inflation of the inflatable cuff.

Example 4 relates to the system of any of Examples 1-3 and 5-15, wherein the inflation of the inflatable cuff causes increased compression of the body part of the patient on which the sleeve is worn.

Example 5 relates to the system of any of Examples 1-4 and 6-15, wherein the tension device comprises a plurality of holes in the sleeve, a string threaded through the plurality of holes, and a motor unit capable of spooling the string.

Example 6 relates to the system of any of Examples 1-5 and 7-15, wherein the motor unit activates upon receiving a communication from the one or more sensors which causes the spooling of the string.

Example 7 relates to the system of any of Examples 1-6 and 8-15, wherein the spooling of the string causes increased compression of the body part of the patient on which the sleeve is worn.

Example 8 relates to the system of any of Examples 1-7 and 9-15, wherein the one or more sensors are motion sensors.

Example 9 relates to the system of any of Examples 1-8 and 10-15, wherein the one or more sensors are gyroscopic sensors.

Example 10 relates to the system of any of Examples 1-9 and 11-15, wherein the one or more sensors are accelerometers.

Example 11 relates to the system of any of Examples 1-10 and 12-15, wherein the one or more sensors are temperature sensors.

Example 12 relates to the system of any of Examples 1-11 and 13-15, wherein the one or more sensors are blood-flow sensors.

Example 13 relates to the system of any of Examples 1-12 and 14-15, wherein the response of the tension device is configurable with an application operating on a mobile device.

Example 14 relates to the system of any of Examples 1-13 and 15, wherein the body part is a leg.

Example 15 relates to the system of any of Examples 1-14, wherein the body part is a waist.

In Example 16, a method for treating a hypotension crisis event comprising equipping a reactive compression device to a patient, the reactive compression device comprising a sleeve shaped to be worn over a body part of a patient, a tension device disposed on the sleeve, and one or more sensors in operable communication with the tension device, and continuously monitoring the patient with the one or more sensors for detection of an orthostatic hypotension event, and wherein the tension device is configured to be actuated in response to a detected orthostatic hypotension event.

Example 17 relates to the method of any of Examples 16 and 18, further comprising a control unit configured to actuate the tension device in response to a detected orthostatic hypotension event.

Example 18 relates to the method of any of Examples 16-17, wherein activating the tension device increases compression on the body part of the patient on which the reactive compression device is worn.

In Example 19, a system for treating a hypotension crisis event comprising a sleeve shaped to be worn over a first body part of a patient, a tension device disposed on the sleeve, a band shaped to be worn over a second body part of a patient, and one or more sensors disposed on the band in operable communication with the tension device.

Example 20 relates to the system of Example 19, wherein the first body part is the lower leg, and the second body part is the upper leg.

While multiple embodiments are disclosed, still other embodiments of the disclosure will become apparent to those skilled in the art from the following detailed description, which shows and describes illustrative embodiments of the invention. As will be realized, the disclosure is capable of modifications in various obvious aspects, all without departing from the spirit and scope of the disclosure. Accordingly, the drawings and detailed description are to be regarded as illustrative in nature and not restrictive.

### BRIEF DESCRIPTION OF DRAWINGS

FIG. 1A shows the system applied to the lower leg of a patient, according to one implementation.

FIG. 1B shows the system with sensors and a sensor band applied to the leg of a patient, according to one implementation.

FIG. 2 shows a diagram of the pump unit of the system, including the pump and control unit, according to one implementation.

FIG. 3 shows the system with a plurality of blood-flow sensors disposed along the sleeve, according to one implementation.

FIG. 4 shows the system with control managed through an application on a mobile device, according to one implementation.

FIG. 5 shows the system with a sleeve applied to the upper leg and an optional pressure relief valve, according to one implementation.

FIG. 6 shows a garment with multiple sleeves and inflatable cuffs, according to one implementation.

FIG. 7 shows a diagram of the system with a sleeve and sensor band configured for use on the torso or midsection, according to one implementation.

FIG. 8 shows the system with multiple sleeves connected by a pneumatic linkage and secured with straps and splints, according to one implementation.

FIG. 9 shows a sleeve having a slit and a string tensioning mechanism, according to one implementation.

FIG. 10 shows the motor unit with a control unit and a motor configured to spool the tensioning string, according to one implementation.

FIG. 11 shows a sleeve joined by a string threaded through holes, with compression applied by a motor unit pulling both ends of the string, according to one implementation.

FIG. 12 shows a sleeve over an underlayer to increase user comfort, according to one implementation.

FIG. 13 shows sleeves over both the thigh and calf of the user, with a shared or individual motor unit, according to one implementation.

FIG. 14 shows a garment with two upper-leg sleeves joined at the bottom and actuated by a motor unit, according to one implementation.

FIG. 15 shows a sleeve made of cross-layered mesh with warps and wefts tensioned by a motor unit, according to one implementation.

FIG. 16 shows a cross-layered mesh garment shaped to fit over the waist of a patient, according to one implementation.

FIG. 17 shows a sleeve with a spiral band tensioned by a string connected to a motor unit, according to one implementation.

FIG. 18 shows a sleeve incorporating electroactive polymers configured to constrict when voltage is applied, according to one implementation.

FIG. 19A shows the electroactive polymer weave in an unactuated state, according to one implementation.

FIG. 19B shows the electroactive polymer weave in a constricted, actuated state, according to one implementation.

FIG. 20A shows a diagram of a wireless electronic communication scheme between the system components, according to one implementation.

FIG. 20B shows a diagram of a wired electronic communication scheme between the system components, according to one implementation.

FIG. 21 shows a flowchart of a method for using the system on a patient, according to one implementation.

FIG. 22 shows the system with non-stretch flaps positioned over structured bladders, according to one implementation.

FIG. 23 shows the system with non-stretch components over structured bladders that are reinforced by asymmetric balloons and supports, according to one implementation.

FIG. 24 shows the system with a garment using non-stretch flaps positioned over structured bladders extending over part of the torso of a user, according to one implementation.

FIG. 25 shows the system with non-stretch flaps positioned over structured bladders positioned on the front or back of a garment, according to one implementation.

### DETAILED DESCRIPTION

Described herein is a system, along with related devices and methods, for providing variable and adaptive compression for the treatment of a hypotension crisis event. The term hypotension crisis event, as used in this disclosure, includes all conditions, diseases, and disorders that relate to a difficulty in returning blood from extremities to the heart. This can include, but is not limited to orthostatic hypotension, postural orthostatic tachycardia syndrome, chronic venous stasis, autonomic dysfunction, peripheral nerve damage, and conditions associated with intravascular hypovolemia.

As those in the art would understand, hypotension crisis events, like orthostatic hypotension events, are often characterized by a drop in blood pressure of a patient when the patient changes posture. It is often noticed when standing up from a seated position, though it can occur in various other situations. Compression clothing can provide some relief to hypotension crisis events, but wearing compression clothing continuously is often uncomfortable. Additionally, the compression clothing provides only a specific amount of compression, based on the tension of the fabric. The disclosed systems, methods, and devices provide comfort and variable compression to users.

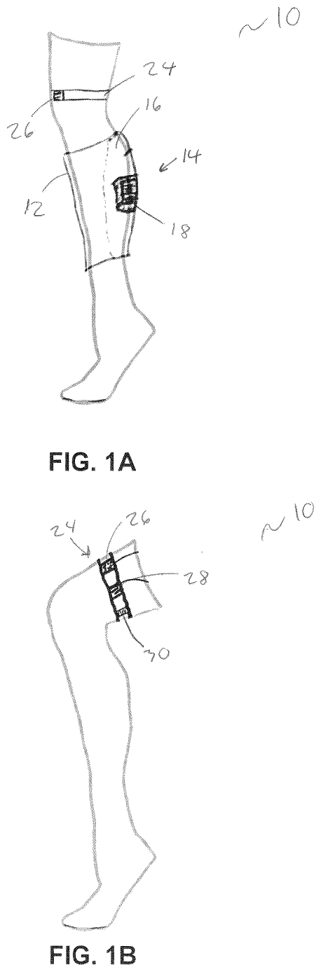

Turning now to FIGS. 1A and 1B, the system 10, in various implementations, may include a sleeve 12 that is substantially cylindrical. In some implementations, the sleeve 12 is shaped to fit over the calf or lower leg of a patient, although other shapes and sizes are possible, such as for use over alternative body parts, as will be discussed below. The sleeve 12 may have a tension device 14, which may have various operating principles, as will be discussed throughout this disclosure. One implementation of the tension device 14 may be an inflatable cuff 16. In various implementations, the inflatable cuff 16 is a bladder that can be filled with fluid which causes the inflatable cuff 16 to enlarge and exert pressure/compression on the body part to which the inflatable cuff is adjacent. In some implementations, the fluid is air, although other fluids are possible. As would be understood, the enlargement of the inflatable cuff 16, when the sleeve 12 is worn by a patient, causes pressure on the body part of the patient on which the sleeve 12 is worn. In the implementation shown in FIGS. 1A and 1B, the body part is the lower leg, although other body parts are possible and would be understood.

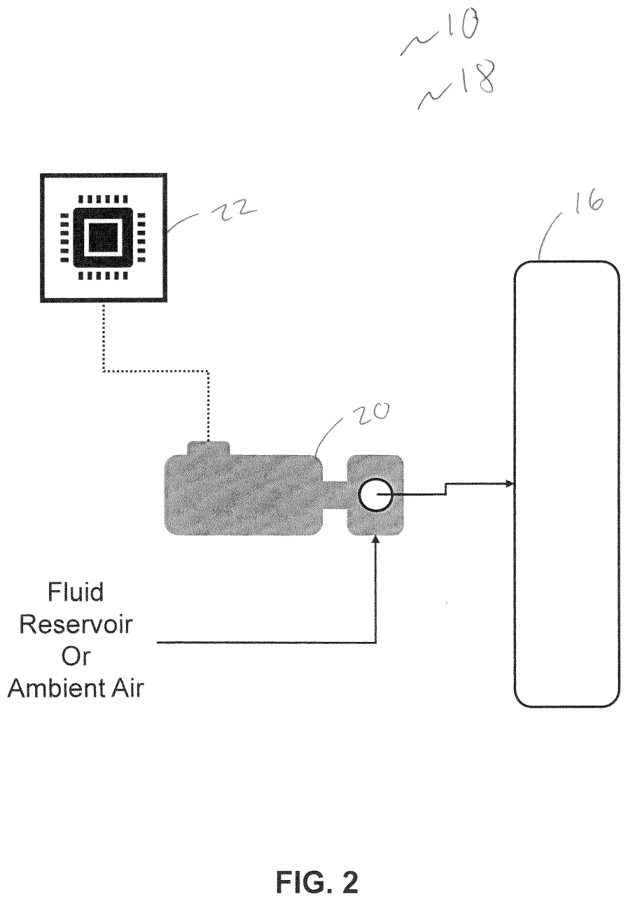

In various implementations, the inflatable cuff 16 is filled with fluid by a pump unit 18. Turning briefly to FIG. 2, the pump unit 18 can, in some implementations, be made of a pump 20 and a control unit 22. The pump 20 may be of any style suitable for pumping fluid against high static pressure, such as a positive displacement pump, though other styles of pump are possible. The control unit 22 may be an electronic device capable of activating and powering the pump, where the activation is in response to one or more inputs, which will be discussed in detail below.

Turning back to FIGS. 1A and 1B, the system 10, in some implementations, may also have a sensor band 24. The sensor band 24 may be a ring of material of appropriate size to hold itself in place on a wearer. Optionally, the sensor band 24 material may be elastic, an elastic blend, or other similar material or fabric type, understood and appreciated by those of skill in the art. In various implementations, the sensor band 24 is worn on the wearer above the knee such that the sensor band 24 moves in space when the wearer moves from a seated to a standing position. In other implementations, the sensor band 24 can be worn on the torso, arms, or other body part. Optionally, the sensor band 24 is worn above the knee such as to be able to detect movement between a sitting and standing position.

In various implementations, the sensor band 24 may have a motion sensor 26. In various implementations, the motion sensor 26 is a device able to detect its own movement and thereby infer motion of the wearer. In some implementations, the motion sensor 26 may be an accelerometer, gyroscope, or other devices capable of detecting their own motion. In various implementations, the motion sensor 26 is in electronic communication with the pump unit 18 and the motion sensor 26 is configured to communicate information about the motion of the motion sensor 26 to the pump unit 18. In some implementations, the pump unit 18 is configured to activate the pump to inflate the inflatable cuff 16 when information from the motion sensor 26 corresponds to the wearer of the sensor band 24 moving from a seated to a standing position. As would be understood, the motion sensor 26 is configured to continuously or intermittently monitor the wearer/patient for information corresponding to a state that indicates a high likelihood of an orthostatic hypotension event in order to provide real-time or near real-time treatment for any such event. That is, for example, when the motion sensor 26 detects that the user has moved from a sitting to a standing position, the pump unit 18 may be activated to provide compression and treatment for a hypotension event nearly instantaneously.

In some implementations, such as shown in FIG. 1B, the sensor band 24 may have a temperature sensor 28 configured to measure the skin temperature of the wearer of the sensor band 24. In such implementations, the temperature sensor 28 may be in electronic communication with the control unit 22 and the temperature sensor 28 is configured to communicate information about the temperature of the skin of the wearer to the control unit 22. In some implementations, the control unit 22 is configured to activate the pump to inflate the inflatable cuff 16 when information from the temperature sensor 28 corresponds to the wearer of the sensor band 24 moving from a seated to a standing position. As would be understood, the skin temperature of a lower extremity, such as a leg, can increase during a hypotension crisis event due to the rush of blood from the torso and head into the lower extremities. As would be understood, the temperature sensor 28 is configured to continuously or intermittently monitor the wearer / patient for information corresponding to a state that indicates a high likelihood of an orthostatic hypotension event in order to provide real-time or near real-time treatment for any such event. That is, for example, when the temperature sensor 28 detects an increase in temperature indicative of movement by the user from a sitting to a standing position, the pump unit 18 may be activated to provide compression and treatment for a hypotension event nearly instantaneously.

In further implementations, also shown in FIG. 1B, the sensor band 24 may have a blood-flow sensor 30 configured to measure blood flow of the wearer of the sensor band 24. In such implementations, the blood-flow sensor 30 may be in electronic communication with the control unit 22 and the blood-flow sensor 30 is configured to communicate information about the blood of the wearer to the control unit 22. In various implementations, the information may include blood pressure, blood oxygenation, blood flow, and similar measurements known in the art.

In various implementations, the blood-flow sensor 30 may use near-infrared spectroscopy to measure blood oxygenation, as would be understood by those in the art, by measuring the relative absorption of different wavelengths of light passing through the body. In some implementations, the blood-flow sensor 30 may detect changes in skin temperature of the patient, whereby changes in pooled blood may be calculated. In various implementations, the blood-flow sensor(s) 30 may use digital plethysmography to measure the local blood volume of the patient, whereby hypotension crisis events may be detected through abnormal changes in blood volume. As would be understood, in many hypotension crisis events, the lower extremities experience an abnormal increase in blood volume. In some implementations, the blood-flow sensors 30 may use laser doppler flowmetry to measure blood circulation changes in tissue. These changes in blood circulation, in some implementations, may then be used to predict or detect a hypotension crisis event.

As would be understood, blood-flow sensor 30 can be used to measure the blood pressure, blood oxygenation, and/or blood flow, which would increase in the extremities as blood from the torso would flow to the lower extremities during a hypotension crisis event. As would be understood, the blood-flow sensor 30 is configured to continuously or intermittently monitor the wearer/patient for information corresponding to a state that indicates a high likelihood of a hypotension crisis event in order to provide real-time or near real-time treatment for any such event.

In various implementations, the control unit 22 may activate the tension device 14 when information from a sensor or sensors 26, 28, 30 corresponds to the wearer of the sensor band 24 experiencing abnormal blood flow that is indicative of a hypotension crisis event. The activation of the tension device 14 may be done in a variety of modalities, such as a pulsatile mode, a ramp-up mode, a ramp-down mode, a cascading mode, or any other such mode or combination thereof known in the art.

A pulsatile mode may be one in which the tension device 14 is activated and deactivated in an alternating fashion, so that compression is applied in periodic pulses.

A ramp-up mode may be one in which the tension device 14 slowly activates so that the degree of compression applied is slowly increased up to a desired amount of compression.

A ramp-down mode may be one in which the tension device 14 activates quickly to apply a desired amount of compression, and then that tension is slowly released to slowly decrease the amount of compression applied.

A cascading mode may be one in which the tension device 14 activates quickly to apply a desired amount of compression and then the applied compression is slowly released. The tension device 14 may then quickly apply compression, but to a new amount of compression, after which the compression may again be slowly released. The new amount of compression may be higher than the proceeding amount in the instance of an ascending cascading mode, or the new amount of compression may be lower than the preceding amount in the instance of a descending cascading mode.

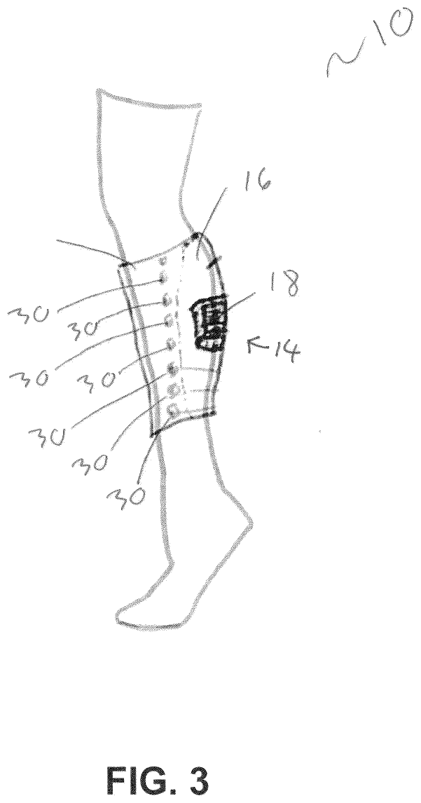

In some implementations, such as shown in FIG. 3, a plurality of blood-flow sensors 30 can be located on a sleeve 12. In various implementations, the plurality of blood-flow sensors 30 are aligned along the sleeve 12 to be substantially parallel with the central axis of the substantially cylindrical sleeve 12. As would be understood, configuring a plurality of blood-flow sensors 30 in this manner allows for the detection of blood flow across the several points of the extremity on which the sleeve 12 is worn. This allows for unusual blood flow to be estimated from the cascading increase of measured blood pressure or oxygenation at each blood-flow sensor 30. Various alternative orientations of the blood-flow sensor 30 are possible and would be understood.

In various implementations, the system 10 may have more than one blood-flow sensor 30, and the blood-flow sensors 30 may employ one or more of the blood flow measurement or detection technologies disclosed herein.

In other implementations, the sensors 26, 28, 30 may all be mounted onto the sleeve 12, rather than being mounted onto a separate sensor band 24.

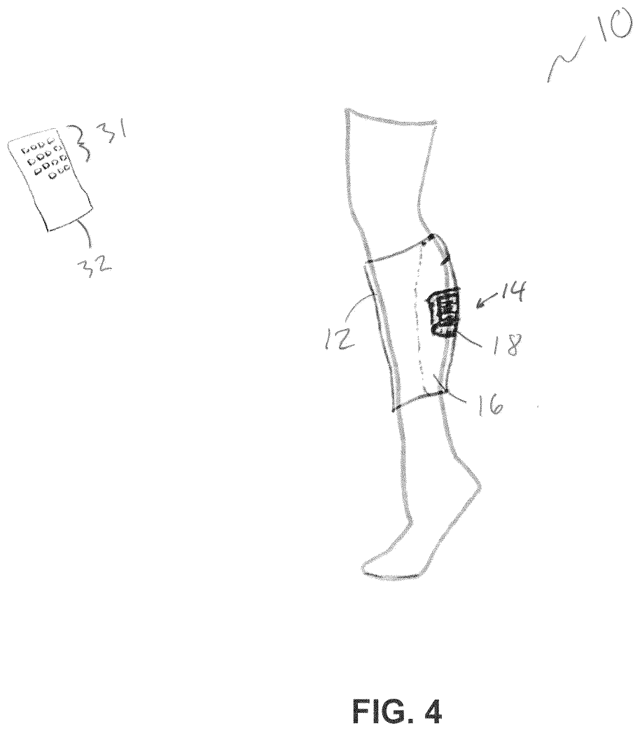

Turning now to FIG. 4, in various implementations, the response of the control unit 22 to electronic communications from one or more sensors 26, 28, 30 may be altered, adjusted, or tuned according to user preference. In some implementations, the response preferences, along with other control details, may be modified through an application 31 on a mobile device 32, such as a cell phone, although other devices are possible. In some implementations, the mobile device 32 and control unit 22 are in electronic communication through a wireless connection, such as a Bluetooth connection, Wi-Fi connection, or similar connection known in the art.

In some implementations, the system 10 may incorporate machine learning and/or artificial intelligence technologies. As would be understood, machine learning and artificial intelligence technologies may be used to improve aspects of the system 10 with repeated use. For instance, in some implementations, a response variable, such as the duration of a hypotension crisis event, may be recorded by the system 10. Similarly, an action variable, such as the degree of tension imparted by the sleeve 12 or a proxy measurement, may be recorded. Numerous other variables, such as the time of day or blood pressure of the patient, may also be recorded. A machine learning or artificial intelligence program may then integrate this data and any other relevant data to create a model for better predicting and responding to hypotension crisis events.

In various implementations, the control unit 22 or mobile device 32 may host the machine learning or artificial intelligence programs. In other implementations, the control unit 22 may share information through the mobile device 32, which is in turn connected to the internet or other wired or wireless communications methods/systems. The mobile device 32 may then use a web-based or cloud-hosted machine learning or artificial intelligence program. In still other implementations, the control unit 22 may be able to be connected directly to the internet and may be able to use web-based or cloud-hosted machine learning or artificial intelligence programs directly. In certain implementations, the system 10 may be integrated or otherwise in communication with electronic medical records.

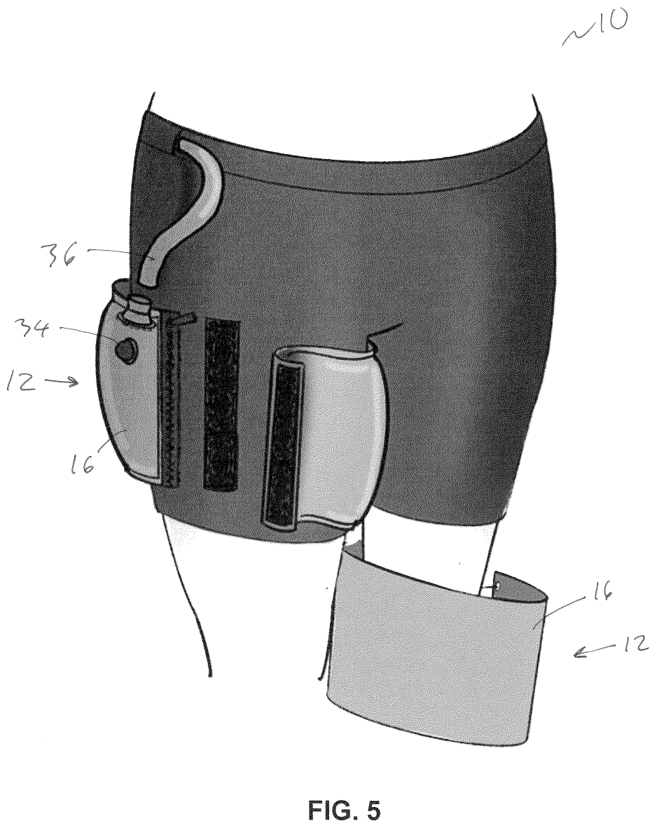

FIG. 5 shows an implementation where one or more sleeves 12 may be shaped to fit to the thigh or upper leg of a user. The sleeves of these implementations may have a tension device 14 that operates using an inflatable cuff 16 and pump unit 18, as described above. Shown in FIG. 5 is an optional pressure relief valve 34 that allows pressurized fluid to exit the inflatable cuff 16 in the event that the inflatable cuff 16 is over-pressurized and risks rupture. The pressure relief valves 34 may also be manual, such that the wearer of the system 10 can deflate the inflatable cuff 16 as desired. The pressure relief valve 34 may be an adjustable relief valve, which allows the wearer to adjust the pressure at which the valves automatically allow pressurized fluid to exit the inflatable cuff 16.

In the implementation shown, the pump unit 18 (not shown) may be remote from the sleeve 12 and may be fluidically connected to the sleeve 12 and inflatable cuff 16 by a supply tube 36.

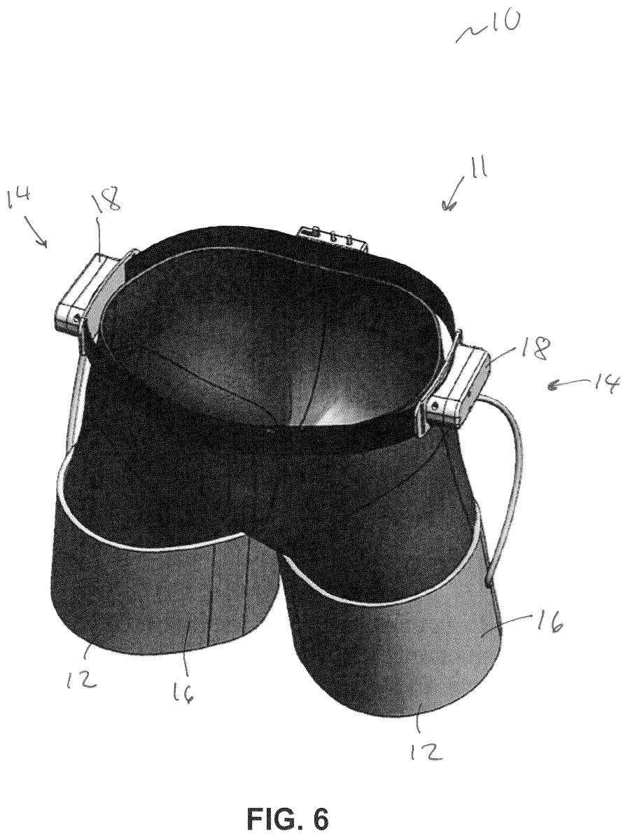

FIG. 6 shows the system 10 using a garment 11, which may have several sleeves 12 disposed thereon. In the implementation shown in FIG. 6, the garment 11 has two sleeves 12, each with an inflatable cuff 16 and corresponding pump unit 18, connected to the inflatable cuff 16 with a supply tube 36. In various implementations, the garment 11 may be an underwear, short, or the like where the sleeves 12 correspond to leg openings.

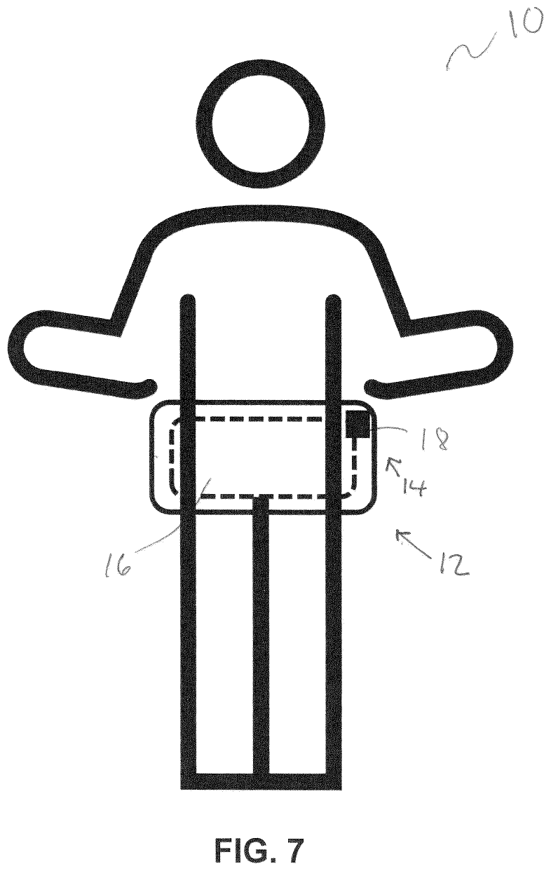

FIG. 7 shows a diagram of an alternative implementation of the system 10 and sleeve 12. The sleeve 12 may be equipped with an inflatable cuff 16 and a pump unit 18, such as is shown in FIGS. 3-6. These implementations function largely as above, wherein a sensor band 24 with various sensors 26, 28, 30 is worn by the patient in a manner that allows the detection of an orthostatic hypotension event, which causes the sensors 26, 28, 30 to communicate the event to the pump unit 18, which inflates the inflatable cuff 16. The inflation of the inflatable cuff 16 around the patient's midsection then prevents the characteristic drop in blood pressure of the head and torso associated with an orthostatic crisis event.

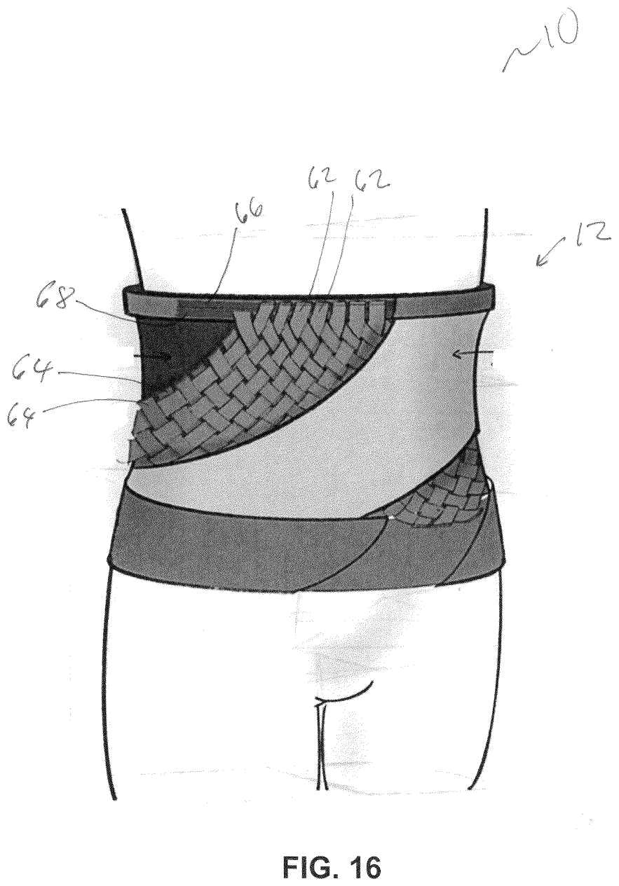

In various implementations, a garment 11 such as is shown in FIG. 6 that may be used primarily for the thighs or posterior of the wearer may be paired with or integrated with the sleeve 12, such as is shown in FIG. 16, which may be used primarily for the waist or trunk of the wearer.

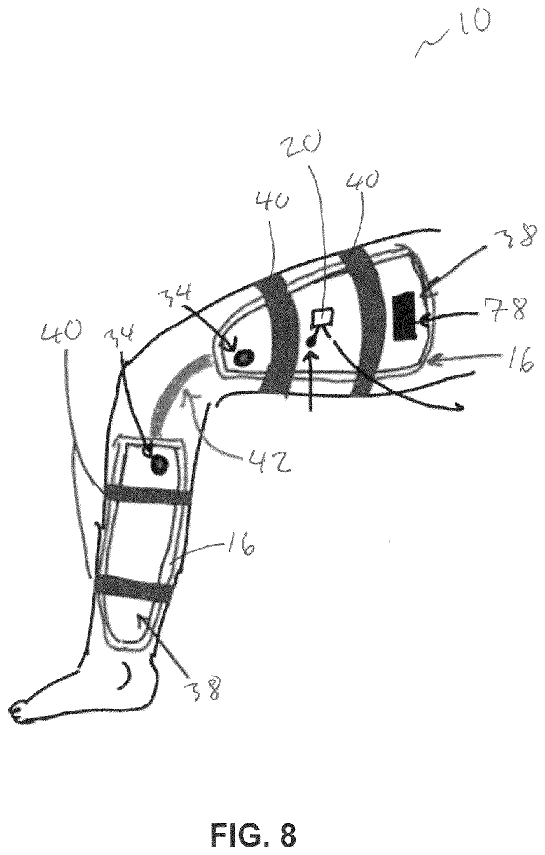

FIG. 8 shows an implementation of the system 10 where the system 10 uses more than one sleeve 12 for a single limb. In such implementations, the several sleeves 12 may be fluidically connected by a pneumatic linkage 42. In various implementations, the pneumatic linkage 42 may be a tube through which fluid may flow between the sleeves 12 in order to keep the fluid pressure of the several sleeves 12 about equal. The several sleeves 12 may be used in combination to provide compression for a variety of body parts at once. For instance, various implementations may provide a sleeve for the lower leg and upper leg, the lower leg and waist, the upper leg and waist, the lower leg, upper leg, and waist, and various other combinations.

The sleeves 12 in FIG. 8 may have splints 38 on the outer surface of the inflatable cuffs 16. The splints 38 may be mostly rigid plates that are curved to match the curvature of the body part for which they are to be connected. The splints 38 and inflatable cuffs 16 may be secured to the relevant body part using one or more straps 40. The straps 40 may be any sort of self-securing belt, such as but not limited to a buckled belt, a Velcro belt, a D-ring belt, hook belt, tie wraps, and the like.

In various implementations, the pneumatic linkage 42 may be flexible enough to allow for movement between the separate sleeves 12 (such as about the knee when the sleeves 12 are on the lower leg and upper leg, or about the hip when the sleeves 12 are on the upper leg and waist), but rigid enough to not crimp, cinch, or otherwise collapse in a way that would restrict airflow.

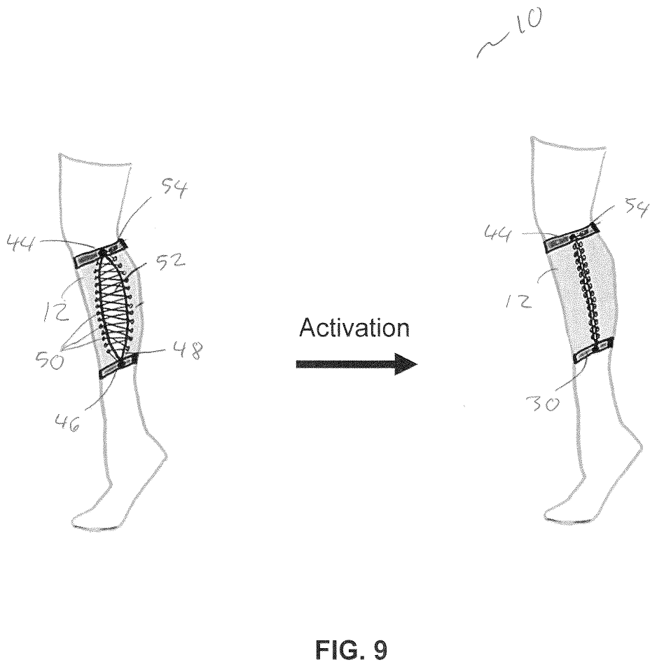

FIG. 9 shows an alternative style of implementations of the sleeve 12. In such implementations, the sleeve 12 may consist of material, optionally fabric, joined onto itself at the top 44 and bottom 46 to create substantially a cylinder with an unjoined slit 48 between the joined top 44 and bottom 46. In certain specific implementations, the sleeve 12 may be made of a tough fabric, such as but not limited to Kevlar.

The sleeve 12 may also have a tension device 14, which in these implementations, consists of the components discussed below. There may be a plurality of holes 50 in the sleeve 12 on either side of the slit 48. Through the plurality of holes 50, a string 52 may be threaded so the slit 48 can be narrowed by pulling on one or both ends of the string 52, substantially like a corset. In some implementations, the string 52 may be anchored to the sleeve 12 at either the top 44 or bottom 46 or to another part of the sleeve 12 that allows for the narrowing of the slit 48 by pulling on an end of the string 52. In such implementations, the sleeve 12 may also have a motor unit 54 configured to draw in the string.

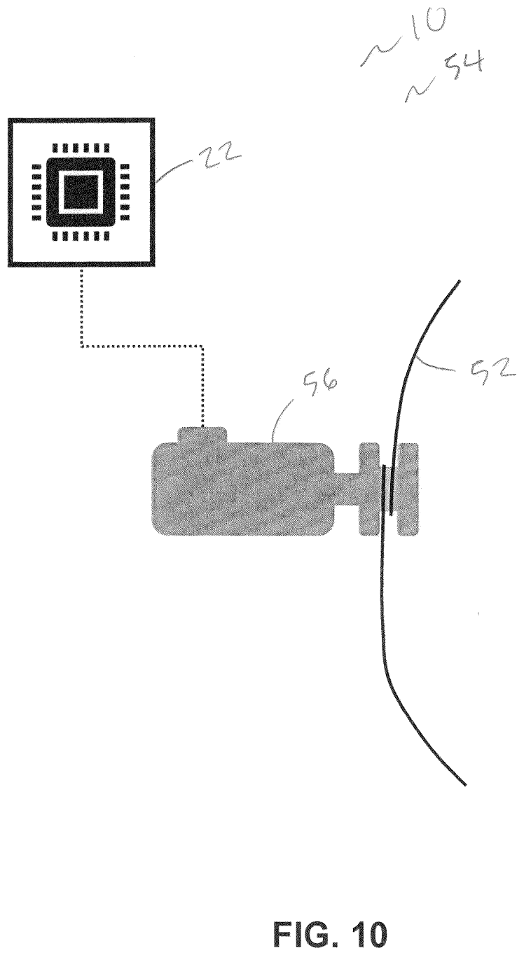

Turning now to FIG. 10, the tension device 14 may have a motor unit 54 that may, in some implementations, contain a motor 56 and a control unit 22. The control unit 22, in these implementations, may behave in substantially the same manner as when coupled with the pump unit 18 and used in a pump unit 18. That manner is that the control unit 22, when contained in a motor unit 54, may still be in electronic communication with one or more motion sensor 26, temperature sensor 28, and/or blood-flow sensor 30, some or all of which may be located on a sensor band 24. These sensors 26, 28, 30 may be configured to detect, through the technologies described above, whether the wearer is changing body position or if the blood of the wearer is flowing abnormally, as would be expected in an orthostatic hypotension event.

In some implementations such as those in FIGS. 9-15, if the motor unit 54 receives the appropriate electronic communication from the relevant sensor or sensors 26, 28, 30, the motor unit 54 may activate the motor 56, which is configured to spool the string 52 around itself. In various implementations, such as those where one end of the string 52 is anchored to the sleeve 12, spooling of the string 52 results in a constricting force to be exerted along the string, which is transferred to the plurality of holes 50, which is subsequently transferred to the sleeve 12. This constricting force causes the slit 48 to narrow, which causes the limb of the wearer of the sleeve to be compressed. As would be understood, this compression, when appropriately applied, can avert or treat a hypotension crisis event.

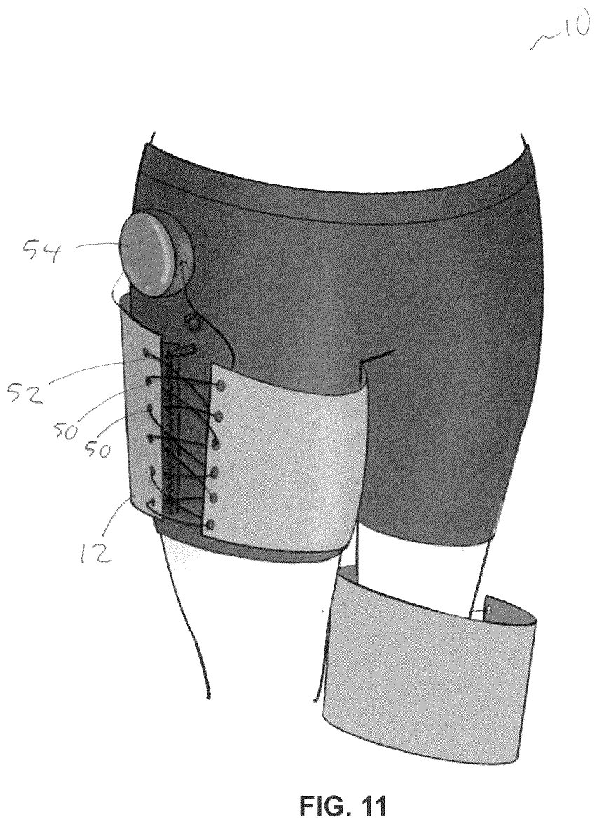

FIG. 11 shows an implementation where the sleeve 12 may consist of material, optionally fabric, joined only through the presence of the plurality of holes 50 and the string 52 threaded therethrough. In the implementation of FIG. 11, the sleeve 12 is shaped to fit around the thigh or upper leg of the user. In this implementation, the motor unit 54 may pull on both ends of the string 52 to draw the slit 48 closed and compress the sleeve 12.

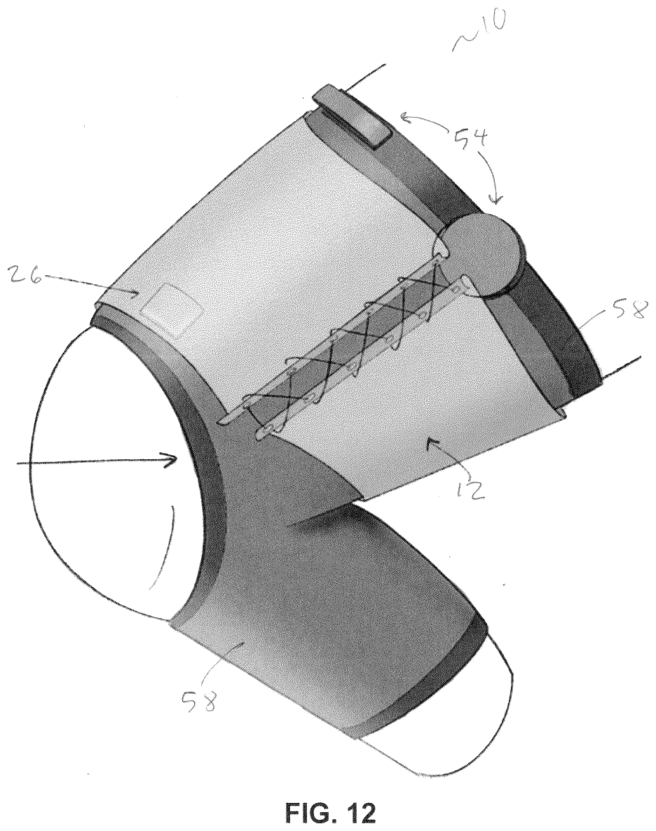

FIG. 12 shows an implementation similar to that of FIG. 11, but the sleeve 12 is set over an underlayer 58, which may allow for more comfort for the user during compression scenarios.

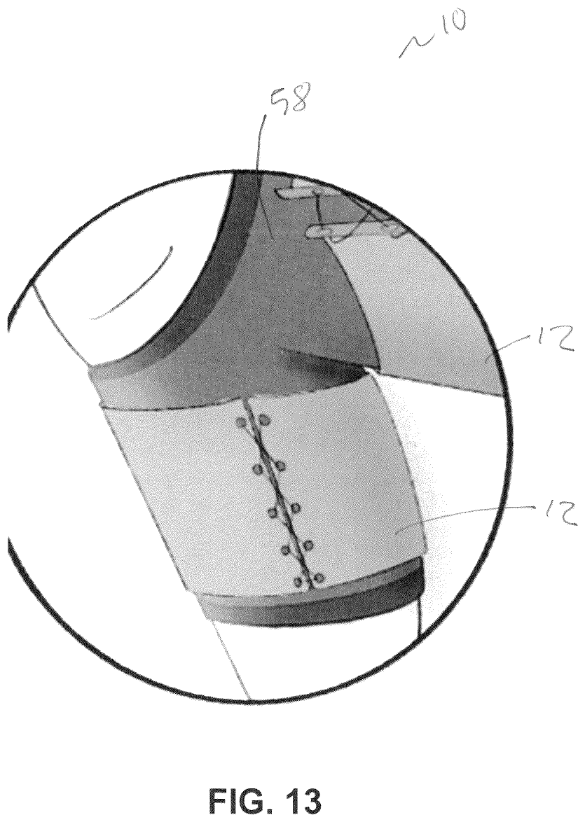

FIG. 13 shows an implementation similar to that of FIG. 12, but with a sleeve 12 over both the thigh and the calf of the user. In such implementations, each sleeve 12 may have its own motor unit 54 and string 52 or a motor unit 54 may be shared between the sleeves, with the string 52 being threaded through both sleeves 12.

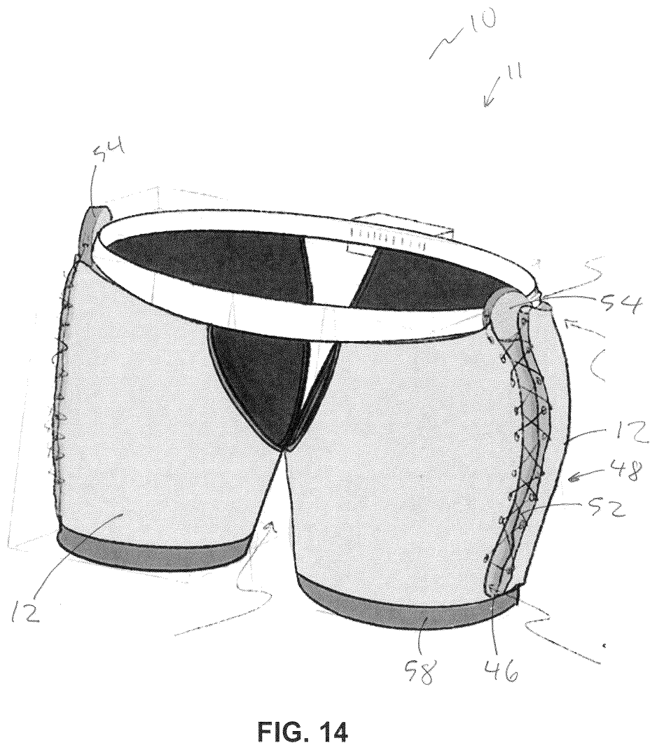

FIG. 14 shows a garment 11 that may consist of two sleeves 12, each shaped to be positioned around the upper leg, such as the thigh and posterior. The sleeves 12 of this implementation may be fixed together at the bottom 46 or may be anchored to an underlayer 58 at the bottom 46, so that the slit 48 is formed above the bottom 46. The string 52 may be threaded through the plurality of holes 50. The motor unit 54, in these implementations, may be configured to draw the slit 48 closed by pulling on the string 52, thus providing compression from the sleeves 12.

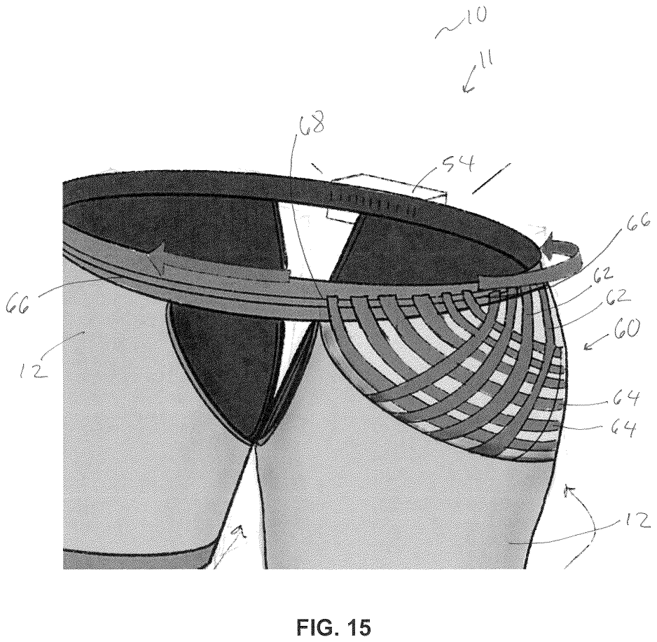

FIG. 15 shows an alternative implementation of the garment 11, where the sleeves 12 are made of a cross-layered mesh 60. The cross-layered mesh 60 may be made of warps 62 and wefts 64, which each may be a piece of material, optionally a thin strand of material. In various implementations, the warps 62 may be attached to a warp string 66 and the wefts may be attached to a weft string 68. The warp string 66 and weft string 68 may be attached to the motor unit 54 such that the motor unit 54 will pull the warp string 66 and weft string 68 in when activated. The warps 62 and wefts 64 may optionally be woven together to form the cross-layered mesh 60 such that when the warp string 66 and weft string 68 are pulled, the warps 62 and wefts 64 create compression, which reduces the overall size of the sleeves 12. As would be understood, this provides compression for the wearer.

The warp string 66 and weft string 68, in addition to tightening the weave of the warps 62 and wefts 64, may also provide compression to the waist and/or thighs of the wearer depending on the location of the garment 11.

FIG. 16 shows an implementation of the garment 11, where the sleeves 12 are made of cross-layered mesh 60 with warps 62 and wefts 64, configured to be drawn tight by a warp string 66 and weft string 68, where the garment 11 is shaped to fit over the waist of a wearer.

In various implementations, a garment 11, such as is shown in FIG. 15, that may be used primarily for the thighs, posterior, and lower waist of the wearer may be paired with or integrated into a garment 11, such as is shown in FIG. 16, that may be used primarily for the waist of the wearer.

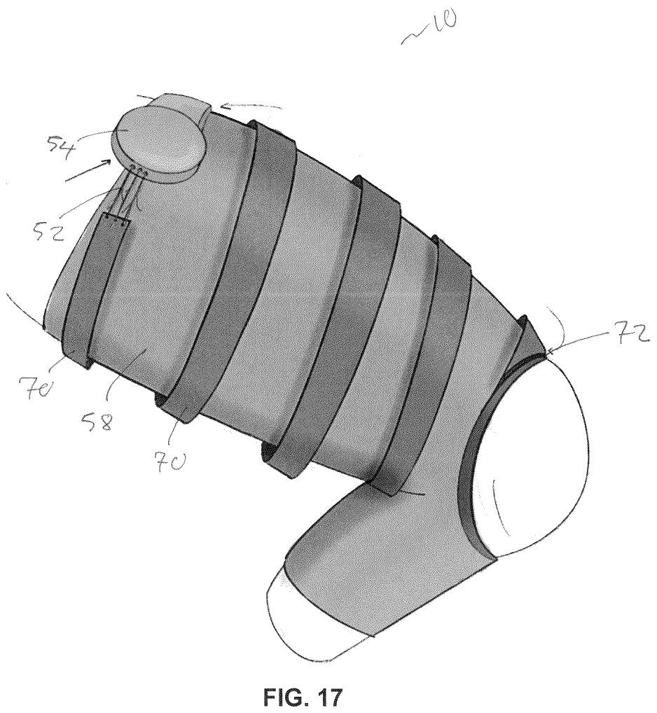

FIG. 17 shows an implementation of the sleeve 12 where the sleeve 12 has an underlayer 58 with a spiral band 70 wrapped around the underlayer 58, with one end of the spiral band 70 anchored to an anchor point 72. The sleeve 12 may have the spiral band 70 shaped to wrap in a spiral pattern around a body part, optionally a thigh or upper leg, of a user when worn. The spiral band 70 may be affixed to a string 52, which may in turn be in operable communication with the motor unit 54. The motor unit 54 may be configured to pull on a string 52, which in turn causes the spiral band 70 to pull tight around the body part of the user, causing compression.

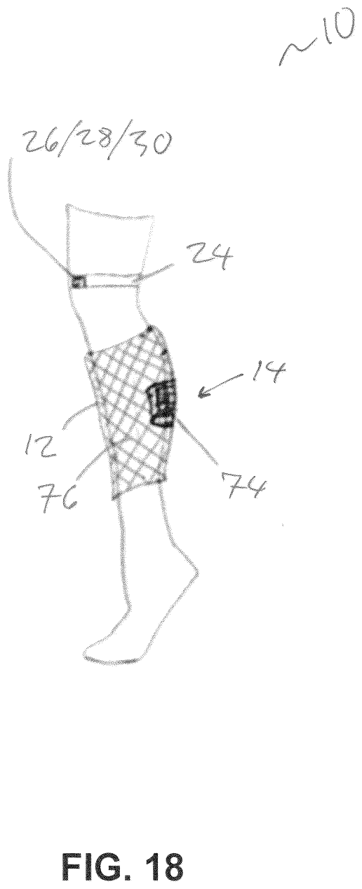

FIG. 18 shows another style of the system 10. In these implementations, the system 10 may incorporate a tension device 14 made of an electroactive polymer into the material of the sleeve 12. As would be understood, electroactive polymers are capable of changing shape or size when stimulated by an electric field or applied voltage. In some implementations, the electroactive polymers in the sleeve 12 are electrically connected to a voltage device 74, which may be a type of tension device 14. In some implementations, the voltage device 74 can have a control unit 22 in operable communication with the sensors 26, 28, 30 and configured to output a voltage or electrical field in the event that the sensors 26, 28, 30 detect a hypotension crisis event. In various implementations, the voltage or electrical field is directed to a weave 76 of electroactive polymers in the sleeve 12, which causes the weave 76 to contract or otherwise deform in a manner that causes a constriction of the sleeve 12.

While FIG. 18 shows the system 10 as a sleeve 12 used over the lower leg of a wearer, the electroactive polymer sleeves 12 may be used on a variety of body parts, such as but not limited to lower legs, upper legs, the posterior, the waist, arms, and the like. The electroactive polymer sleeves 12 may also be used in garments 11 that may provide compression for several body parts, such as but not limited to the upper legs, posterior, and waist.

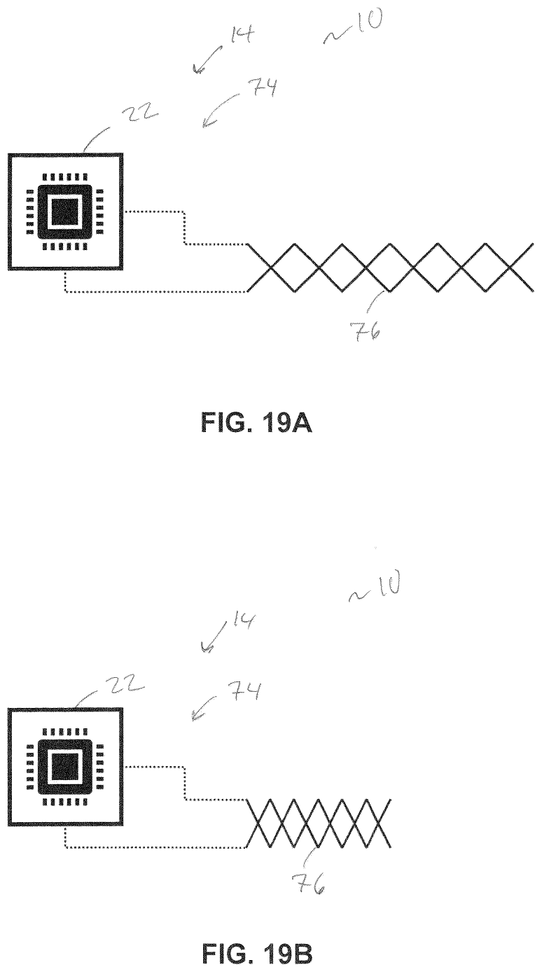

FIG. 19A shows a diagram of the voltage device 74 electrically connected to the weave 76 of electroactive polymers, such as would be in the sleeve 12, but no voltage or electric field is applied to the weave 76. Because no voltage or electric field is applied, the weave 76 is at its baseline shape and size.

FIG. 19B shows a diagram of the voltage device 74 electrically connected to the weave 76, where a voltage or electric field is being applied to the weave 76. In this implementation, the weave 76 then constricts in response to the voltage or electric field.

Similar implementations that are worn over the waist, or other body parts, are also possible with tension devices of any style contemplated herein.

Various other implementations of the system 10 may be configured to provide reactive compression to other body parts, as would be understood. In these various implementations, the system 10 could be designed to provide reactive compression to the feet, lower leg, upper leg, whole leg, waist, hands, forearms, upper arms, whole arms, and various other body parts that may require such treatment.

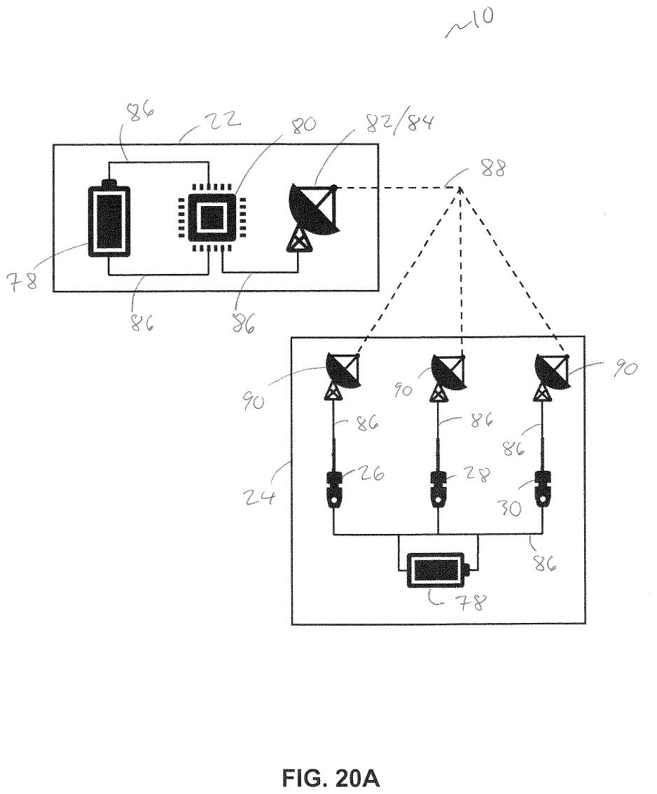

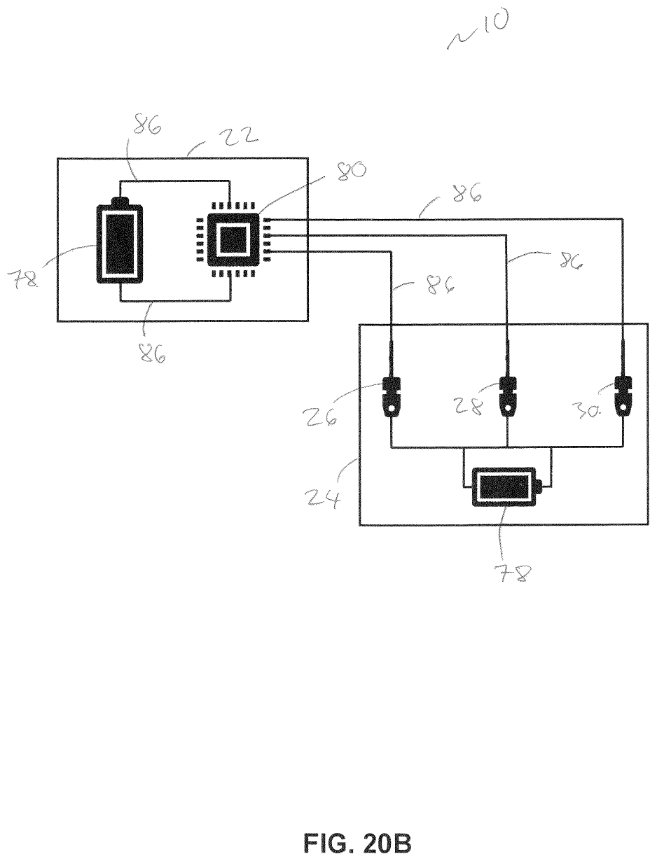

FIG. 20A shows a diagram, according to various implementations, of an electronic communications scheme between the components of the system 10. In such implementations, the control unit 22 may consist of a power source 78 electrically connected to a logic device 80, which is in turn in electronic communication to wireless communications device 82, such as a transceiver 84. The electronic communication may be achieved through wires 86. The power source 78 is capable of providing electrical power, such as 24V DC power, to the logic device 80 and/or the wireless communications device 82. In other implementations, the power source 78 may provide electrical power through other sources, such as through capacitors, or through a direct electrical connection to a power grid. The logic device 80 may be a device capable of receiving electrical signal inputs, performing computations and/or algorithms, and sending electrical signal output, such as a programmable circuit board, microcontroller, microprocessor, or similar device known in the art. In some implementations, the wireless communications device 82 is configured to send output signals and receive input signals to and from the logic device 80 using a wired connection, and the wireless communications device 82 can convert those wired signals to wireless signals 88. In various implementations, the wireless signals are sent and received using Bluetooth, Wi-Fi, or a similar connection known in the art.

The electronic communications scheme of FIG. 20A also shows the sensors 26, 28, 30 disposed on the sensor band 24. In some implementations, each sensor 26, 28, 30 can be electrically connected to a power source 78 through wires 86. Each sensor 26, 28, 30 may then be electrically connected to an individual transmitter 90 configured to send wireless signals 88 to the wireless communications device 82. These wireless signals, as would be understood, convey information from the sensors 26, 28, 30 about the various measured parameters used to detect an orthostatic hypotension event.

FIG. 20B shows a diagram, according to various implementations, of a different electronic communications scheme between the components of the system 10. This electronic communications scheme is similar to the scheme of FIG. 20A, except that rather than wireless signals 88 conveyed from a transmitter 90 to an electrical communications device 82, the sensors 26, 28, 30 may be connected directly through wires 86 to the logic device 80.

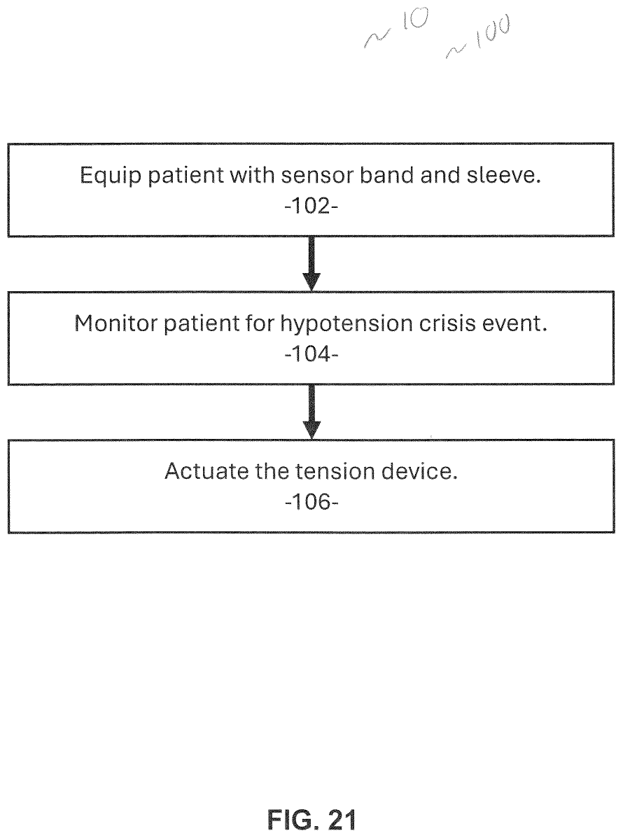

FIG. 21 is a flow chart depicting a method 100 of applying the system 10 to a patient. While these steps are listed and shown in a particular order, the steps given can be performed in any order. Additionally, each step is optional and can be performed or omitted as necessary. A patient may be equipped with a sensor band 24, optionally applied to their upper leg or torso, and the patient may be equipped with a sleeve 12, optionally applied to their lower leg or waist (box 102). The control unit 22 and sensors 26, 28, 30 may then monitor the patient for signs of an orthostatic hypotension event (box 104). If a hypotension crisis event is detected by the sensors 26, 28, 30, the control unit 22 may actuate the tension device 14 (box 106). As would be understood in the art, the actuation of the tension device 14 would cause an increase in compression on the patient body part, such as a leg, inserted into the sleeve. This compression may provide a local increase in blood pressure in that body part, which may prevent blood from elsewhere in the patient, such as the head, from rushing into the body part.

In the various implementations described herein, the system 10 may return the tension device 14 to its original state once the compression is no longer necessary or desired. In some implementations, the system 10 may determine that compression is no longer necessary or desired and thereby return the tension device 14 to its original state upon the expiration of a timer. In such implementations, the timer may begin once the sensors 26, 28, 30 detect a hypotension crisis event and may run for a predetermined time. In other implementations, the system 10 may determine that compression is no longer necessary or desired and thereby return the tension device 14 to its original state once the sensors 26, 28, 30 no longer detect signs of the hypotension crisis event. In still further implementations, the system 10 may activate the tension device 14 once a hypotension crisis event is detected, begin a timer once the sensors 26, 28, 30 no longer detect the hypotension crisis event, and then return the tension device 14 to its original state once that time expires. In various other implementations, other algorithms and logic schemes may be used to determine when the system 10 should relieve compression after applying the compression through the tension device 14.

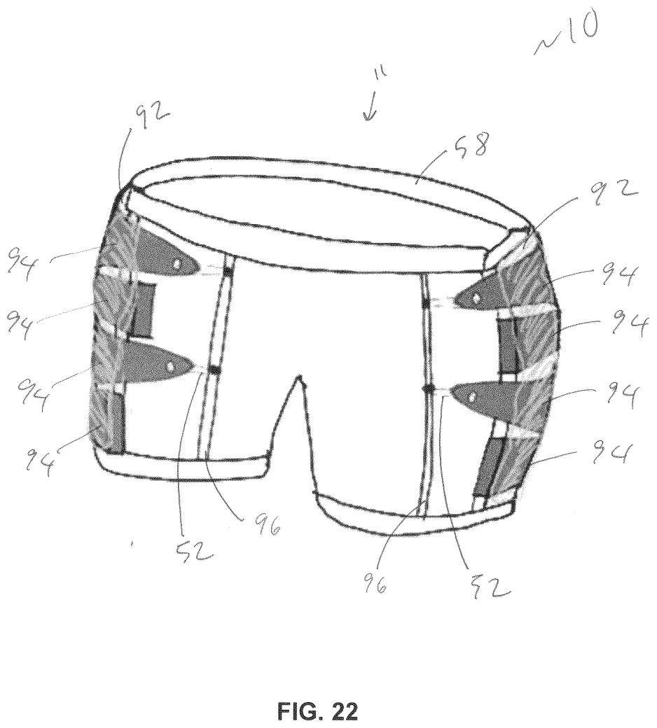

Turning now to FIGS. 22-25, various implementations may use non-stretch components to provide even pressure to a limb or body part during the actuation of the system 10. As will be seen, providing compression to a body part through tension induced into non-stretch components of a garment 11 may provide more even and consistent pressure in the instance of a hypotension crisis event. Reliance on direct compression from the inflation of air or fluid bladders may not fully utilize the energy input into the inflated bladder, and may not adequately provide compression in key areas where there may be little room to position a bladder, such as, but not only, the inner thigh.

FIG. 22 shows one implementation of the system 10 having a garment 11 designed to fit over the posterior and upper thighs of a user. The garment 11 of this implementation may use one or more structured bladders 92. The structured bladders 92 may be positioned in various positions on the garment 11, such as but not limited to over the hips, thighs, posterior, lower legs, waist, stomach, chest, and arms.

The structured bladders 92 may be fixed to an underlayer 58 The structured bladders 92 may be inflatable balloons that expand substantially in one direction. For instance, in some implementations, the structured bladders 92 may have non-stretch sides on some sides while having stretchable sides elsewhere. This may allow the structured bladders 92 to expand substantially in one direction, while maintaining their dimensions with respect to all other dimensions.

In some implementations, the structured bladders 92 may be positioned between the underlayer 58 and one or more non-stretch flaps 94. The non-stretch flaps 94 may be attached to the underlayer 58 through one or more strings 52, optionally at one or more seams 96 in the underlayer 58. While different configurations of non-stretch flaps 94 may be used, some implementations have the non-stretch flaps 94 oriented in alternating orientation, allowing some non-stretch flaps 94 to connect to the rear of the underlayer 58 and other non-stretch flaps 94 to the front of the underlayer 58.

In various implementations, the structured bladders 92 may be inflated, such as by the pump unit 18, which may expand laterally outwardly substantially in one direction. In other implementations, the structured bladders 92 may expand in variety of other directions or may expand outwardly in all directions. The outward expansion of the structured bladders 92 may force the non-stretch flaps 94 apart and away from the underlayer 58. Due to the non-stretch nature of the non-stretch flaps 94, and the attachment of the non-stretch flaps 94 at each end to the underlayer 58, the non-stretch flaps 94 may pull on the underlayer 58. As the underlayer 58 is pulled, it may create tension through the underlayer 58 that causes the underlayer 58 to cinch down on the body parts of the user. This cinching action created through tension may result in compression of the body parts of the user under the garment 11. In some implementations, the underlayer 58 may be non-stretch or may have patches of non-stretch material to better transfer the tension delivered through the strings 52 and non-stretch flaps 94 into compression of the body part.

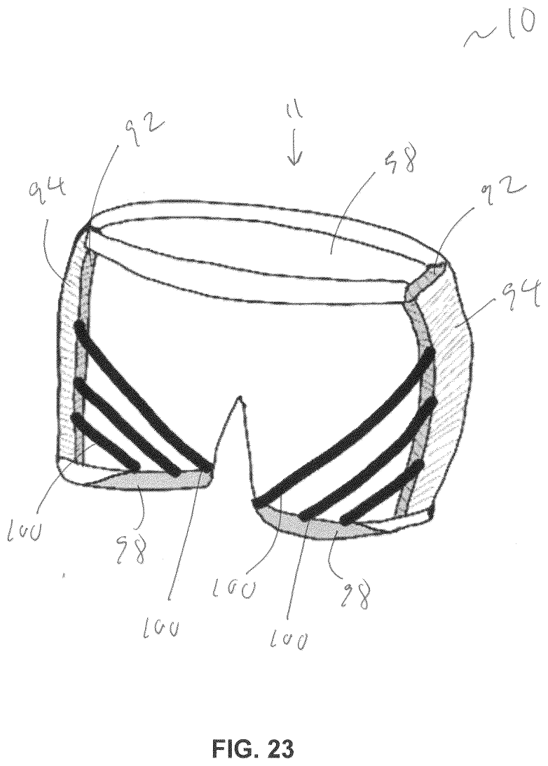

Turning now to FIG. 23, in some implementations, the garment 11 of the system 10 may use a single non-stretch flap 94 for each structured bladder 92, with each non-stretch flap 94 positioned overtop of each structured bladder 92, relative to the underlayer 58. Each non-stretch flap 94 of these implementations may attach, such as through stitching, to the underlayer 58, or various other portions of the garment 11, such that the inflation of the structured bladder 92 causes tension through the underlayer 58 or garment 11, which thereby creates compression on the body part of the user.

In various implementations, the garment 11 may have one or more asymmetric balloons 98 positioned on the garment 11. In some implementations, these asymmetric balloons 98 may be positioned on the garment 11 to be approximately perpendicular to the structured bladders 92. The garment 11 may have to one or more supports 100 running between the asymmetric balloons 98 and the structured bladders 92. The asymmetric balloons 98 and supports 100 may prevent the structured bladders 92 from bulging outward during inflation, allowing the expansion of the structured bladders 92 to transfer more tension into the garment 11, increasing compression on the body part. In various implementations, the asymmetric balloons 98 may be constructed such that they expand substantially only in one direction, such as laterally.

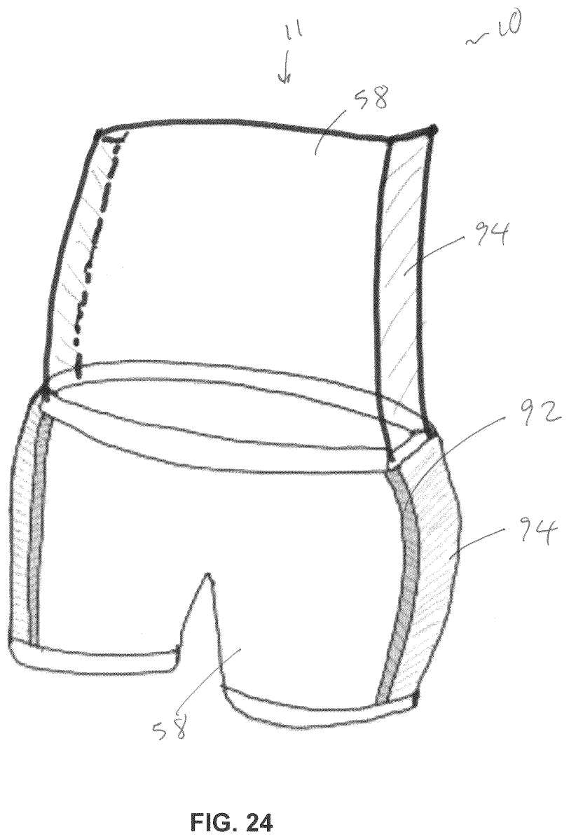

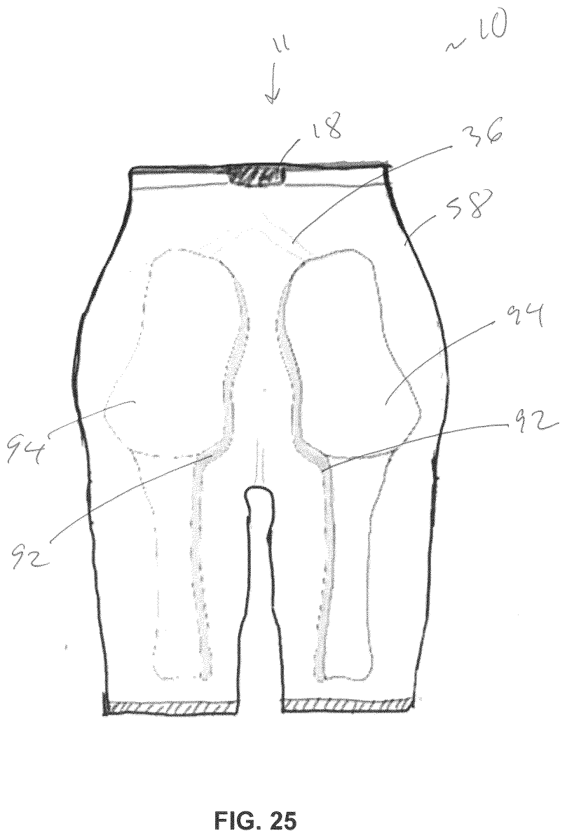

FIG. 24 shows an implementation of the system 10 where the garment 11 may be shaped to fit over the thighs and posterior of a user, as well as extending upwards to cover the waist, stomach, and lower torso generally. In implementations like this, the structured bladders 92 and non-stretch flaps 94 may also extend upwards to provide compression to the torso of the user.

FIG. 25 shows an implementation of the system 10 with a garment 11 having structured bladders 92 positioned on the front or rear of the garment 11, relative to the perspective of the wearing user. FIG. 25 also shows the pump unit 18 in fluidic communication with the structured bladders 92 through one or more supply tubes 36 that may allow the pump unit 18 to force fluid, such as air, into the structured bladders 92 in order to inflate them.

Although the disclosure has been described with reference to various implementations, persons skilled in the art will recognize that changes may be made in form and detail without departing from the spirit and scope of the disclosure.

What is claimed is:

1. A system for treating a hypotension crisis event comprising:
(a) a garment;
(b) a structured bladder constructed to expand when filled with a fluid; and
(c) a non-stretch component positioned to impart tension on the garment when the structured bladder expands.

2. The system of claim 1, wherein the non-stretch component is a flap connected to the garment with a string.

3. The system of claim 2, wherein the string is connected to a seam in the garment.

4. The system of claim 1, further comprising a pump unit in fluidic communication with the structured bladder.

5. The system of claim 4, further comprising a sensor in operable communication with the pump unit, the sensor configured to detect a hypotension crisis event.

6. The system of claim 5, wherein the pump unit is configured to inflate the structured bladder when the sensor detects a hypotension crisis event.

7. A garment for treating a hypotension crisis event comprising:
(a) an underlayer shaped to be worn over a body part of a patient;
(b) a structured bladder constructed to expand when filled with a fluid;
(c) a non-stretch component positioned to impart tension on the underlayer when the structured bladder expands; and
(d) a pump unit constructed to pump fluid into the structured bladder.

8. The garment of claim 7, wherein the structured bladder is positioned between the underlayer and non-stretch component.

9. The garment of claim 7, wherein the structured bladder comprises at least one non-stretch side and at least one stretchable side.

10. The garment of claim 7, further comprising one or more asymmetric balloons positioned on the garment.

11. The garment of claim 10, wherein the one or more asymmetric balloons are approximately perpendicular to the structured bladder.

12. The garment of claim 11, further comprising one or more supports connecting the structured bladder and asymmetric balloons.

13. The garment of claim 7, wherein the non-stretch component comprises a series of flaps.

14. The garment of claim 13, wherein each flap of the series of flaps alternates in orientation.

15. The garment of claim 7, wherein the non-stretch component comprises a single flap constructed to substantially cover the structured bladder.

16. A method of treating a hypotension crisis event comprising:
equipping a patient with a garment, the garment comprising:
(i) a structured bladder constructed to expand when filled with a fluid; and
(ii) a non-stretch component positioned to impart tension on the garment when the structured bladder expands;
monitoring the patient with one or more sensors configured to detect a hypotension crisis event; and
inflating the structured bladder in response to a detected hypotension crisis event, wherein expansion of the structured bladder causes the non-stretch component to impart tension on the garment to provide compression to a body part of the patient.

17. The method of claim 16, wherein the garment further comprises an underlayer positioned between the garment and the body part, and the structured bladder is positioned between the underlayer and the non-stretch component.

18. The method of claim 16, wherein inflating the structured bladder forces the non-stretch component away from an underlayer of the garment, causing the non-stretch component to pull on the underlayer to create compression on the body part.

19. The method of claim 16, wherein the sensor measures temperature.

20. The method of claim 16, wherein the sensor measures blood-flow.
